(19) United States
(12) Patent Application Publication
(10) Pub. No.: US 2021/0077244 A1
(43) Pub. Date: Mar. 18, 2021

(54) STENT-GRAFTS COMBINATION FOR CENTRAL VEINS

(71) Applicant: Shin-Kong Wu Ho-Su Memorial Hospital, Taipei City (TW)

(72) Inventor: Chia-Hsun LIN, Taipei City (TW)

(21) Appl. No.: 16/571,757

(22) Filed: Sep. 16, 2019

(51) Int. Cl. A61F 2/07; A61F 2/90

(52) U.S. Cl. CPC A61F 2/07; A61F 2/88; A61F 2/90

(57) ABSTRACT

A stent-grafts combination comprising a first stent and a second stent for central veins is provided, in which the first stent and the second stent with appropriate diameters are selected depending on patient's veins and overlappingly sleeve-coupled with each other. The stent-graft for central veins can improve the matching of the stent-graft with different patients' veins, so that the surgical success rate is increased and the postoperative problems caused by the inappropriate diameters of stent-graft is decreased.

### BACKGROUND

### Technology Field

The present disclosure is related to a stent-graft for vessels, especially, a stent-grafts combination for central veins.

### Description of Related Art

For a long time, a patient must undergo kidney dialysis treatment when he or she progressed to the process of chronic renal failure. In kidney dialysis or other treatments, an intubation via the central venous catheter is often needed for the patient to facilitate the kidney dialysis treatment or other treatment. However, based on clinical experience, about 25% to 40% of patients suffered from central venous stenosis or obstruction in chronic renal dialysis patients. Since the venous obstruction occurs near the heart, the entire hand, forearm and upper arm will gradually develop severe swelling and discomfort, even the skin is ulcerated and the wound is difficult to heal. Until now, the main therapy for central venous obstruction is intravascular balloon angioplasty. However, the recurrent stenosis is a common postoperative sequela, since at this time, the vascular wall loses its elasticity, becomes fibrotic and tends to intimal hyperplasia.

Therefore, the treatment of venous obstruction necessitates to use a stent-graft for mechanical support and material barrier. Furthermore, each patient has different diameters at the proximal and distal ends of the central vein due to the individual differences, so that the treatment for venous obstruction should select a stent-graft which matches with the size of vessel in the patient's affected region. A cardiovascular stent-graft supplier usually provides standardized stent-grafts in order to avoid the increased manufacturing costs and storage burden. Thus, most patients cannot obtain the standardized stent-grafts suitable for matching the diameters at both ends of the vessels in their affected regions due to the individual differences and the venous obstruction in the different position. If the standardized stent-grafts that does not completely match with the diameter at both ends of the vessel, it may cause various postoperative complications caused by the unsuitable stent-graft, such as dislodgment due to undersized proximal end or restenosis due to oversized distal end. Otherwise, it takes two stent-grafts with different sizes for overlap and results in additional cost. Therefore, it is an important issue to find a stent-grafts that is suitable for matching the diameters of the blood vessels in the various patients.

The present disclosure provides a stent-grafts combination comprising a first stent-graft and a second stent-graft. The first stent-graft and the second stent-graft with suitable diameters can be selected in accordance with the size of vessel in a patient's affected region, and may be sleeve-coupled overlappingly each other. Accordingly, the stent-grafts combination of the present disclosure can improve the mis-matching of the stent-graft with the vessels in the different positions of different patients, reduce postoperative complications, decrease the producers' storage burden and the medical cost.

### SUMMARY

In order to solve such problem, the present disclosure provides a stent-graft for central veins comprising a first stent-graft including a proximal section and a sleeve-coupling section as well as a second stent-graft including a sleeve-coupling section and a distal section, wherein a diameter of an end portion of the proximal section of the first stent-graft is larger than that of an end portion of the distal section of the second stent. The first stent-graft and the second stent-graft are artificial vascular stent-grafts, and a tubular structure having the different diameters at both end portions can be formed after sleeve-coupling the first stent-graft with the second stent-graft each other.

In the stent-grafts for central veins provided by the present disclosure, the diameter of the end portion of the proximal section of the first stent-graft is not particularly limited and can be manufactured in accordance with the size of vein in the patient's affected region, as long as the blood stream can be directed out from the first stent-graft and does not leak out from a contact surface between the first stent-graft and vascular wall. In one embodiment, the diameter of the end portion of the proximal section of the first stent-graft is preferably form 14 mm to 20 mm.

In the stent-graft for central veins provided by the present disclosure, the diameter of the end portion of the distal section of the second stent-graft is not particularly limited and can be manufactured in accordance with the size of vein in the patient's affected region, as long as the blood stream can be directed out from the second stent-graft and does not leak out from a contact surface between the second stent-graft and vascular wall. In one embodiment, the diameter of the end portion of the distal section of the second stent-graft is preferably form 8 mm to 16 mm.

The artificial vascular cover of the first stent-graft and the second stent-graft in the present disclosure is composed of a tubular artificial vascular wrapped by a plurality of elastically supported metal stents. The artificial vascular cover may be a human body acceptable high-molecular-weight medical material, and the conventional medical material is a film or fabric manufactured by, for example, polytetrafluorethylene, polyamide, polyester fiber, polypropylene and the like. The elastically supported metal stent is manufactured by a human body acceptable alloy, such as Nitinol, nickel-titanium elastic alloy, medical stainless steel alloy, the alloy comprising iron, cobalt, nickel, chromium, molybdenum and the like. In addition, the shape of the elastically supported metal stent may be annular, spiral, wavy or reticular formation.

In the stent-graft for central veins provided by the present disclosure, the second stent-graft is placed in the upstream of the first stent-graft, and the sleeve-coupling section of the second stent-graft can be sleeve-coupled into sleeve-coupling section of the first stent-graft. A tubular structure having the different diameters at both ends can be formed after the sleeve-coupling of the first stent-graft with the second stent-graft each other. The diameters of two sleeve-coupling sections are not particularly limited as long as it is easy to operate during the surgical procedure and they are fully attached each other after sleeve-coupling the first stent-graft with the second stent-graft such that the blood stream is not leaked out.

In one embodiment, the sleeve-coupling section of the first stent-graft or the sleeve-coupling section of the second stent-graft has a uniform diameter, and each diameter is from 10 to 14 mm, and the diameter difference between the sleeve-coupling section of the first stent-graft and the sleeve-coupling section of the second stent-graft is preferably from 2 to 3 mm. In one embodiment, the diameter at the sleeve-coupling section of the first stent-graft is less than that at the sleeve-coupling section of the second stent-graft, and thus the sleeve-coupling section of the second stent-graft is overlappingly sleeve-coupled to the inner side of the sleeve-coupling section of first stent-graft. Since the second stent-graft has a larger diameter, the sleeve-coupling sections of two stent-grafts can be completely attached each other via the expandable elasticity of the elastically supported metal stent the first stent-graft and the second stent-graft after the overlappingly connection into the sleeve-coupling section of the first stent-graft. In another embodiment, the diameter at the sleeve-coupling section of the first stent-graft is greater than that at the sleeve-coupling section of the second stent-graft, and thus the sleeve-coupling section of the first stent-graft is sleeve-coupled into the inner side of the sleeve-coupling section of the second stent-graft.

In one embodiment, after sleeve-coupling the first stent-graft with the second stent-graft each other, the length of the overlapping portion can be determined by the factors such as the length of the sleeve-coupling sections, the difference in the diameters of two stent-graft, and the total length after sleeve-coupling in the condition that two stent-graft are completely attached each other without leaking blood. For example, the length of the overlapping portion can be determined by a person ordinarily skilled in the art according to the diameter difference between the sleeve-coupling sections of the first stent-graft and the second stent-graft. If the diameter difference between the sleeve-coupling sections of the first stent-graft and the second stent-graft is smaller, the completely overlapping of two sleeve-coupling sections can be considered. In another embodiment, the determination of the total length of the intended stent-graft formed by sleeve-coupling the first stent-graft with the second stent-graft based on the patient's need, and the length of the overlapping portion can be regulated according to the total length of said intended stent-graft. In another embodiment, the lengths of the sleeve-coupling sections of the first stent-graft and the second stent-graft are from 10 to 30 mm, respectively, and preferably, each is 20 mm. In another embodiment, the overlapping portion of two sleeve-coupling sections is 20 mm after sleeve-coupling the first stent-graft with the second stent-graft.

In the stent-graft for central veins provided by the present disclosure, the first stent-graft and the second stent-graft may have a same or different length. In other words, a person ordinarily skilled in the art can understand that the length of the first stent-graft and the second stent-graft can be selected and sleeve-coupled appropriately after assessing the factors such as the vascular structure, size, extent, ease of surgery, safety and the like in the patient's affected region. In one embodiment, after sleeve-coupling the first stent-graft with the second stent-graft, the total length is 80 mm or more, and preferably, from 80 to 200 mm. In another embodiment, after sleeve-coupling the first stent-graft with the second stent-graft, the total length is 80, 85, 90, 95, 100, 105, 110, 115, 120, 125, 130, 135, 140, 145, 150, 155, 160, 165, 170, 175, 180, 185, 190, 195 or 200 mm.

In the stent-graft for central veins provided by the present disclosure, the external surfaces of the first stent-graft and/or the second stent-graft may be further provided a plurality of fixed members for fixing to the vascular wall, and the fixed member can be a barbed or anchor structure. By such way, a plurality of fixed members can increase the anchoring force of the first stent-graft and/or the second stent-graft to the vascular wall, and contribute to the accuracy and stability of disposing the stents. In another embodiment, the fixed members are disposed at the outer periphery of the end portion of the distal section of the second stent-graft. In another embodiment, the fixed members can be further disposed at the outer periphery of the end portion of the sleeve-coupling section of the first stent-graft if the second stent-graft is sleeve-coupled into the inner side of the first stent-graft.

The stent-graft for central veins according to the present disclosure can be used to treat a variety of vascular lesions, such as vascular obstruction, hemangioma, vascular disease, etc. For example, in the case of treating a single obstruction of blood vessels, the first stent-graft and the second stent-graft can be sleeve-coupled each other and disposed through the single stenosis in the blood vessel. If a section of blood vessel exists or will possibly exist multiple obstructions, the first stent-graft and the second stent-graft can be sleeve-coupled each other and disposed through the section of blood vessel. In the case of treating a hemangioma, after the sleeve-coupling, the proximal section of the first stent-graft is disposed at the inner side of neck of proximal end of the hemangioma, and the distal section of the second stent-graft is disposed at the inner side of neck of distal end of the hemangioma, such that the first stent-graft and the second stent-graft are sleeve-coupled each other and disposed through the both ends of hemangioma.

As used herein, the articles “a” and “an” refer to one or to more than one (i.e., to at least one) of the grammatical object of the article. By way of example, “a fixed member” means one fixed member or more than one fixed member.

As used herein, the term “human body acceptable” means that it usually does not cause an allergic reaction, rejection, inflammatory response or the like after the application to a human body.

As used herein, the terms “upstream” and “downstream” of blood vessel are determined by the direction of blood stream. In the case of veins, since the venous blood stream from the peripheral vein to the heart, at one relative position of the blood vessel, such as at the vascular obstruction, the side of peripheral vein is referred to “upstream”, and the side of the heart is referred to “downstream”. In the case of arteries, since the arterial blood stream from the heart to the peripheral artery, at one relative position of the blood vessel, such as at the vascular obstruction, the side of the heart is referred to “upstream” and the side of peripheral artery is referred to “downstream”.

It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 8 to 14 should be considered to have specifically disclosed subranges such as from 8 to 9, from 8 to 10, from 8 to 11, from 9 to 10, from 9 to 11, from 10 to 14, etc., as well as individual numbers within that range, for example, 8, 9, 10, 10.5, 11, 12.6, 13.2 and 14. This applies regardless of the breadth of the range.

### BRIEF DESCRIPTION OF THE DRAWINGS

FIG. 1 is a schematic diagram of the stent-grafts combination for central veins according to present disclosure.

FIG. 2 is a schematic diagram of sleeve-coupling overlappingly the first stent-graft with the second stent-graft.

FIG. 3 is another schematic diagram of the stent-grafts combination for central veins according to present disclosure.

FIG. 4 is another schematic diagram of sleeve-coupling overlappingly the first stent-graft with the second stent-graft.

FIG. 5 is a schematic diagram of disposing the stent-grafts combination of the present disclosure in a blood vessel.

### DETAILED DESCRIPTION OF THE DISCLOSURE

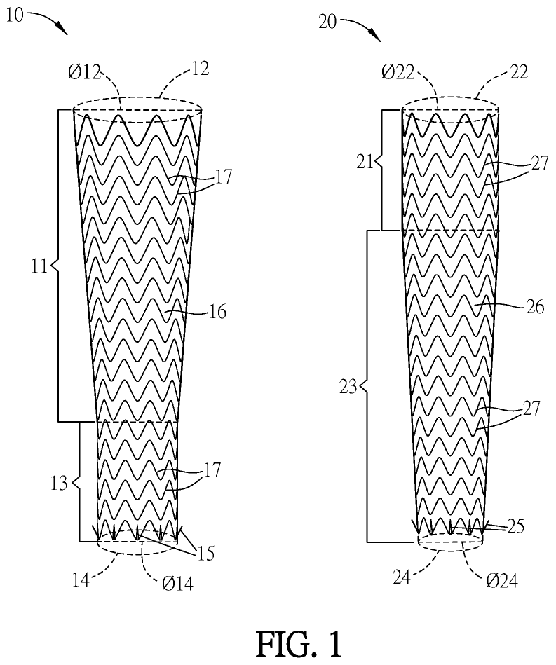

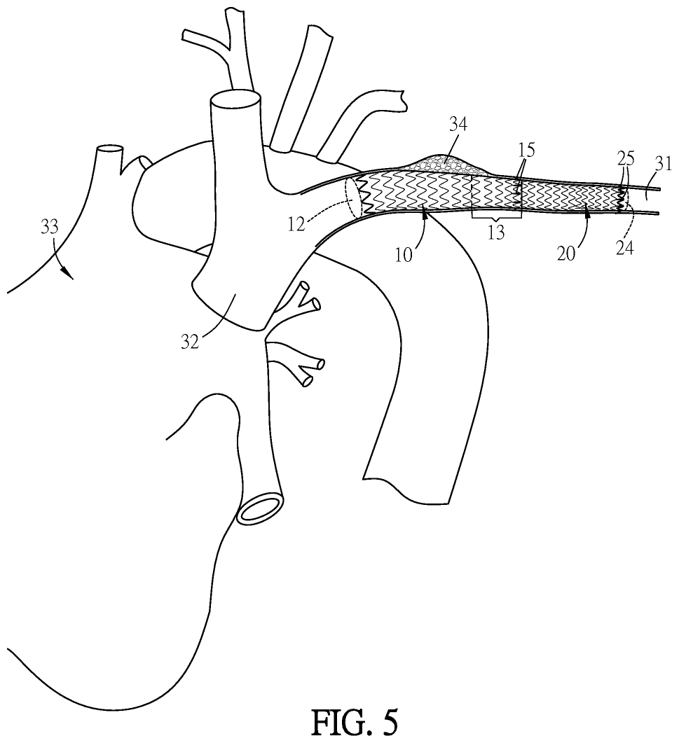

The examples of the stent-grafts combination for central veins according to the present disclosure is illustrated in detail with reference to FIG. 1˜FIG. 5. These examples are provided for the purpose of illustration only, and the disclosure is not limited to these examples

### Example 1

Referring to FIG. 1, a schematic diagram of the stent-grafts combination for central veins according to present disclosure is shown. The stent-graft for a central vein according to present disclosure is consisted of a first stent-graft 10 and a second stent-graft 20, and the first stent-graft 10 and the second stent-graft 20 are thin round tubes composed of an artificial vascular cover 16, 26 on which several elastically supported metal stents 17, 27 are weaved.

The first stent-graft 10 includes a proximal section 11 and a sleeve-coupling section 13. The proximal section has an end portion 12 with a diameter ϕ12. The sleeve-coupling section 13 has a uniform diameter, and an end portion 14 thereof has a diameter ϕ14. A plurality of fixed members 15 for fixing to the vascular wall are disposed at the outer periphery of the end portion 14 of the sleeve-coupling section.

The second stent-graft 20 includes a sleeve-coupling section 21 and a distal section 23. The sleeve-coupling section 21 has a uniform diameter, and the end 22 thereof has a diameter ϕ22. The distal section 23 has an end portion 24 with a diameter ϕ24, and a plurality of fixed members 25 for fixing to the vascular wall are disposed at the outer periphery of the end portion 24 of the distal section.

The diameter ϕ12 of the end portion of the proximal section of the first stent-graft is 16 mm, and the diameter ϕ24 of the end portion of the distal section of the second stent-graft is 8 mm.

### Example 2

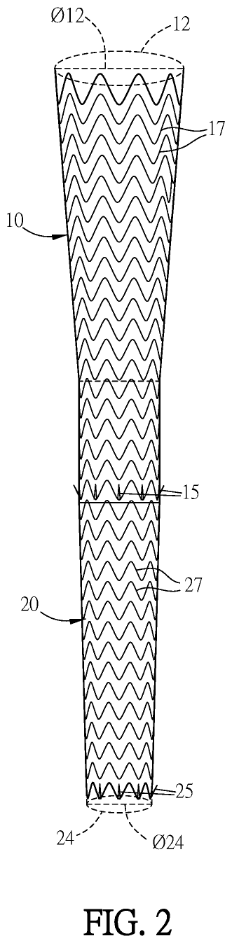

Referring to FIG. 1 and FIG. 2, FIG. 2 is a schematic diagram of the stent-grafts combination for central veins according to the present disclosure after sleeve-coupling overlappingly the first stent-graft with the second stent-graft each other. The stent-grafts combination for central veins according to the present disclosure is substantially a round tube that tapers gradually from the proximal section 11 of the first stent-graft to the distal section 23 of the second stent-graft after sleeve-coupling overlappingly the first stent-graft 10 with the second stent-graft 20 each other. When the sleeve-coupling section 21 of the second stent-graft is sleeve-coupled into the sleeve-coupling section 13 of the first stent-graft from the end portion 14, the overlapping portion of the sleeve-coupling section 13 and the sleeve-coupling section 21 is about 20 mm. In addition, the diameter ϕ14 of the end portion 14 of the sleeve-coupling section of the first stent-graft must be smaller than the diameter ϕ22 of the end portion 22 of the sleeve-coupling section of the second stent-graft, and the difference between the diameter ϕ14 and the diameter ϕ22 is about 2 mm. Since the diameter ϕ22 of the end portion of the sleeve-coupling section of the second stent-graft is slightly larger than the diameter ϕ14 of the end portion 14 of the sleeve-coupling section of the first stent-graft, when the second stent-graft is sleeve-coupled into the first stent-graft, the outer surface of the sleeve-coupling section 21 of second stent-graft is completely attached to the inner side of the sleeve-coupling section 13 of the first stent-graft via the expandable elasticity of the elastically supported metal stents 17, 27, such that the blood leaking can be prevented efficiently. It is advantageous for the long-term use of the stent-grafts combination for central veins according to the present disclosure.

### Example 3

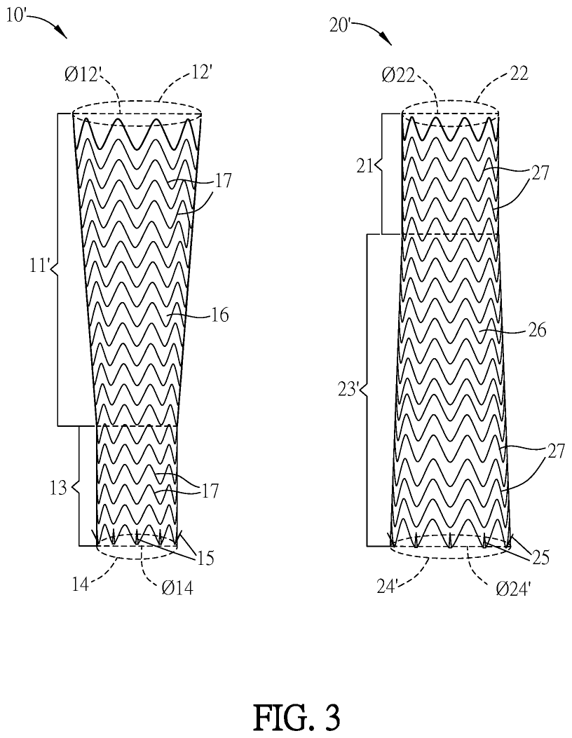

Referring to FIG. 3, another schematic diagram of the stent-grafts combination for central veins according to present disclosure is shown. The stent-grafts for a central vein is consisted of a first stent-graft 10′ and a second stent-graft 20′, and the first stent-graft 10′ and the second stent-graft 20′ are thin round tubes composed of an artificial vascular cover 16, 26 on which several elastically supported metal stents 17, 27 are weaved.

The first stent-graft 10′ includes a proximal section 11′ and a sleeve-coupling section 13. The proximal section has an end 12′ with a diameter 12′. The sleeve-coupling section 13 has a uniform diameter, and the end thereof has a diameter ϕ14. A plurality of fixed members 15 for fixing to the vascular wall are disposed at the outer periphery of the end portion 14 of the sleeve-coupling section.

The second stent-graft 20′ includes a sleeve-coupling section 21 and a distal section 23′. The sleeve-coupling section has a uniform diameter, and the end portion 22 thereof has a diameter 22. The distal section has an end portion 24′ with a diameter 24′, and a plurality of fixed members 25 for fixing to the vascular wall are disposed at the outer periphery of the end portion 24′ of the distal section.

The diameter ϕ12′ of the end portion of the proximal section of the first stent-graft is 20 mm, and the diameter ϕ24′ of the end portion of the distal section of the second stent-graft is 16 mm.

### Example 4

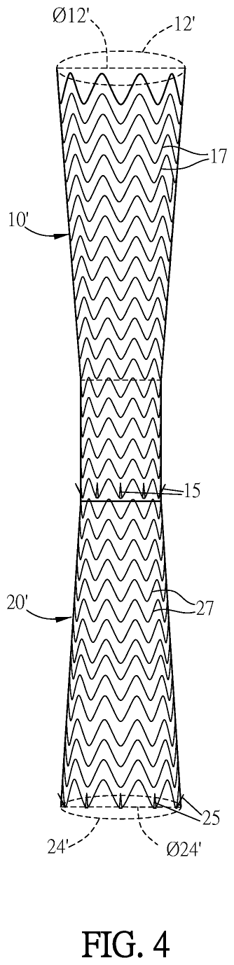

Referring to FIG. 3 and FIG. 4, FIG. 4 is another schematic diagram of the stent-grafts combination for central veins according to the present disclosure after sleeve-coupling overlappingly the first stent-graft with the second stent-graft each other. The stent-grafts combination for central veins according to the present disclosure is substantially a round tube after sleeve-coupling overlappingly the first stent-graft 10′ with the second stent-graft 20′ each other. When the sleeve-coupling section 21 of the second stent-graft is sleeve-coupled into the sleeve-coupling section 13 of the first stent-graft from the end portion 14, the overlapping portion of the sleeve-coupling section 13 and the sleeve-coupling section 21 becomes about 20 mm. In addition, the diameter ϕ14 of the end portion 14 of the sleeve-coupling section of the first stent-graft must be smaller than the diameter ϕ22 of the end portion 22 of the sleeve-coupling section of the second stent-graft, and the difference between the diameter ϕ14 and the diameter ϕ22 is about 2 mm. Since the diameter ϕ22 of the end portion of the sleeve-coupling section of the second stent-graft is slightly larger than the diameter ϕ14 of the end portion 14 of the sleeve-coupling section of the first stent-graft, when the second stent-graft is sleeve-coupled into the first stent-graft, the outer surface of the sleeve-coupling section 21 of second stent-graft is completely attached to the inner side of the sleeve-coupling section 13 of the first stent-graft via the expandable elasticity of the elastically supported metal stents 17, 27, such that the blood leaking can be prevented efficiently. It is advantageous for the long-term use of the stent-grafts combination for central veins according to the present disclosure.

### Example 5

Referring to FIG. 1 and FIG. 5, FIG. 5 is a schematic diagram of disposing the stent-grafts combination of the present disclosure in a blood vessel. In the procedure of disposing the stent-grafts combination, a first stent-graft with a suitable diameter ϕ12 of the end portion of the proximal section is first selected depending on the vascular diameter at the downstream of vascular obstruction 34 in the patient's left brachiocephalic vein 31, and the first stent-graft 10 is set at the downstream of vascular obstruction 34 by a stent-graft delivery device (not shown). The first stent-graft can be fixed to an intended position by a plurality of fixed members 15 disposed at the outer periphery of the end portion 14 of the sleeve-coupling section, such that the displacement caused by blood stream can be avoided. Next, a second stent-graft 20 with a suitable diameter ϕ24 of the end portion of the distal section is selected depending on the vascular diameter at the upstream of vascular obstruction 34 in the patient's left brachiocephalic vein 31. The sleeve-coupling section 21 of the second stent-graft is sleeve-coupled into the sleeve-coupling section 13 by a stent-graft delivery device (not shown), so that the second stent-graft is set at the upstream of vascular obstruction 34. The second stent-graft can be fixed to an intended position by a plurality of fixed members 25 disposed at the outer periphery of the end 22 of the distal section. After setting the stent-grafts for a central vein according to present disclosure in place, the blood can be introduced into the stent-grafts from the end portion 24 of distal section of second stent-graft and into superior vena cava 32, and flows through the vascular obstruction 34 without hindrance, and then flow out of the end 12 of proximal section of the first stent-graft and into the heart 33. Since a plurality of fixed members 15, 25 are disposed, there is no risk of displacement or being flushed into the heart 33 by blood stream.

What is claimed is:

1. A stent-grafts combination for central veins comprising
a first stent-graft, which is an artificial vascular stent-graft, including a proximal section and a sleeve-coupling section, wherein a diameter of an end portion of the proximal section of the first stent-graft is 16 mm or more, and a diameter of an end portion of the sleeve-coupling section of the first stent-graft is from 10 to 14 mm; and
a second stent-graft, which is an artificial vascular stent-graft, including a sleeve-coupling section and a distal section, wherein a diameter of an end portion of the sleeve-coupling section of the second stent-graft is from 10 to 14 mm, a diameter of an end portion of the distal section of the second stent-graft is 16 mm or less, the sleeve-coupling section of the second stent-graft is sleeve-coupled with the sleeve-coupling section of the first stent-graft, and a diameter difference between the end portions of the two sleeve-coupling sections is from 2 to 3 mm;
wherein the diameter of the end portion of the proximal section of the first stent-graft is larger than the diameter of the end portion of the distal section of the second stent-graft.

2. The stent-grafts combination for central veins of claim 1, wherein an overlapping length of sleeve-coupling parts of the sleeve-coupling sections of the second stent-graft and the first stent-graft is from 10 to 30 mm.

3. The stent-grafts combination for central veins of claim 1, wherein the sleeve-coupling section of the first stent-graft has a uniform diameter.

4. The stent-grafts combination for central veins of claim 2, wherein the sleeve-coupling section of the first stent-graft has a uniform diameter.

5. The stent-grafts combination for central veins of claim 1, wherein the sleeve-coupling section of the second stent-graft has a uniform diameter.

6. The stent-grafts combination for central veins of claim 2, wherein the sleeve-coupling section of the second stent-graft has a uniform diameter.

7. The stent-grafts combination for central veins of claim 1, wherein an overlapping length of sleeve-coupling parts of the sleeve-coupling sections of the second stent-graft and the first stent-graft is 20 mm.

8. The stent-grafts combination for central veins of claim 1, wherein the diameter of the end portion of the proximal section of the first stent-graft is from 16 to 20 mm.

9. The stent-grafts combination for central veins of claim 1, wherein the diameter of the end portion of the distal section of the second stent-graft is from 8 to 14 mm.

10. The stent-grafts combination for central veins of claim 1, wherein the diameter of the end portion of the distal section of the second stent-graft is ≥14 mm˜16 mm, when the diameter of the end portion of the proximal section of the first stent-graft is ≥18 mm˜20 mm.

11. The stent-grafts combination for central veins of claim 1, wherein the diameter of the end portion of the distal section of the second stent-graft is greater than 8 mm and less than 14 mm, when the diameter of the end portion of the proximal section of the first stent-graft is greater than 16 mm and less than 18 mm.

12. The stent-grafts combination for central veins of claim 1, wherein the total length is from 80 to 200 mm after sleeve-coupling the first stent-graft with the second stent-graft.

13. The stent-grafts combination for central veins of claim 1, wherein a plurality of fixed members for fixing to vascular wall are disposed at an outer periphery of the end portion of the distal section of the second stent-graft.

14. The stent-grafts combination for central veins of claim 1, wherein a plurality of fixed members for fixing to vascular wall are disposed at an outer periphery of the end portion of the sleeve-coupling section of the first stent.
